(19) United States
(12) Patent Application Publication
(10) Pub. No.: US 2012/0135949 A1
(43) Pub. Date: May 31, 2012

(54) COMBINATION THERAPY FOR TREATING HCV INFECTION

(75) Inventors: Wulf Otto BOECHER, Biberach (DE); Carla HAEFNER, Neu-Ulm (DE); George KUKOLJ, Mount Royal (CA)

(73) Assignee: BOEHRINGER INGELHEIM INTERNATIONAL GMBH, Ingelheim am Rhein (DE)

(21) Appl. No.: 13/241,613

(22) Filed: Sep. 23, 2011

Related U.S. Application Data
(60) Provisional application No. 61/388,253, filed on Sep. 30, 2010.

(51) Int. Cl. A61K 31/506; A61P 31/14; A61K 31/7056

(52) U.S. Cl. 514/43; 514/256

(57) ABSTRACT

The present invention relates to therapeutic combinations comprising (a) Compound (1), or a pharmaceutically acceptable salt thereof, as herein described, (b) Compound (2), or a pharmaceutically acceptable salt thereof, as herein described, and optionally (c) ribavirin, and methods of using such therapeutic combinations for treating HCV infection or alleviating one or more symptoms thereof in a patient.

### TECHNICAL HELD OF THE INVENTION

The present invention relates to therapeutic combinations comprising Compounds (1) and (2) as herein described and optionally ribavirin. The present invention also relates to methods of using such therapeutic combinations for treating HCV infection or alleviating one or more symptoms thereof in a patient.

### BACKGROUND OF THE INVENTION

Hepatitis C virus (HCV) infection is a global human health problem with approximately 150,000 new reported cases each year in the United States alone. HCV is a single stranded RNA virus, which is the etiological agent identified in most cases of non-A, non-B post-transfusion and post-transplant hepatitis and is a common cause of acute sporadic hepatitis. It is estimated that more than 50% of patients infected with HCV become chronically infected and 20% of those develop cirrhosis of the liver within 20 years.

Several types of interferons, in particular, alfa-interferons are approved for the treatment of chronic HCV, e.g., interferon-alfa-2a (ROFERON®-A), interferon-alfa-2b (INTRON®-A), consensus interferon (INFERGEN®), as well as pegylated forms of these and other interferons like pegylated interferon alfa-2a (PEGASYS®) and pegylated interferon alfa-2b (PEG-INTRON®). Most patients are unresponsive to interferon-alfa treatment, however, and among the responders, there is a high recurrence rate within 6 months after cessation of treatment (Liang et al., J. Med. Virol. 40:69, 1993).

Ribavirin, a guanosine analog with broad spectrum activity against many RNA and DNA viruses, has been shown in clinical trials to be effective against chronic HCV infection when used in combination with interferon-alfas (see, e.g., Poynard et al., Lancet 352:1426-1432, 1998; Reichard et al., Lancet 351:83-87, 1998), and this combination therapy has been approved for the treatment of HCV: REBETRON® (interferon alfa-2b plus ribavirin, Schering-Plough); PEGASYS®RBV® (pegylated interferon alfa-2a plus ribavirin combination therapy, Roche); see also Manns et al, Lancet 358:958-965 (2001) and Fried et al., 2002, N. Engl. J. Med. 347:975-982. However, even with this combination therapy the virologic response rate is still at or below 50%.

Furthermore, there are significant side-effects typically associated with such therapies. Ribavirin suffers from disadvantages that include teratogenic activity, interference with sperm development, haemolysis, fatigue, headache, insomnia, nausea and/or anorexia. Interferon alfa, with or without ribavirin, is associated with many side effects. During treatment, patients must be monitored carefully for flu-like symptoms, depression, rashes and abnormal blood counts. Patients treated with interferon alfa-2b plus ribavirin should not have complications of serious liver dysfunction and such subjects are only considered for treatment of hepatitis C in carefully monitored studies.

The following Compound (1):

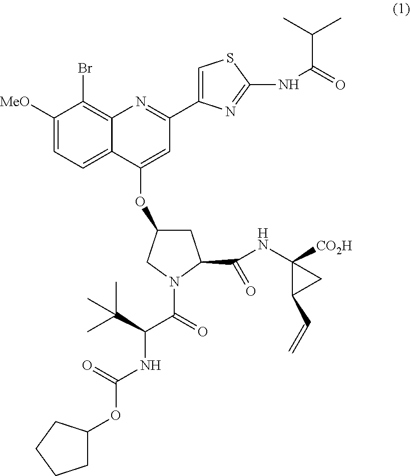

having the chemical name: 1-{[4-[8-Bromo-2-(2-isopropylcarbamoyl-thiazol-4-yl)-7-methoxy-quinolin-4-yloxy]-1-(R)-(2-cyclopentyloxycarbonyl amino-3,3-(S)-dimethyl-butyryl)-pyrrolidine-(S)-2-carbonyl]-amino}-2-(S)-vinyl-cyclopropane-(R)-carboxylic acid, is known as a selective and potent inhibitor of the HCV NS3 serine protease and useful in the treatment of HCV infection. Compound (1) falls within the scope of the acyclic peptide series of HCV inhibitors disclosed in U.S. Pat. Nos. 6,323,180, 7,514,557 and 7,585,845. Compound (1) is disclosed specifically as Compound #1055 in U.S. Pat. No. 7,585,845, and as Compound #1008 in U.S. Pat. No. 7,514,557. Compound (1), and pharmaceutical formulations thereof, can be prepared according to the general procedures found in the above-cited references, all of which are herein incorporated by reference in their entirety. Preferred forms of Compound (1) include the crystalline forms, in particular the crystalline sodium salt form as described in U.S. Patent Application Publication No. 2010/0093792, also incorporated herein by reference.

Compound (1) may also be known by the following alternate depiction of its chemical structure, which is equivalent to the above-described structure:

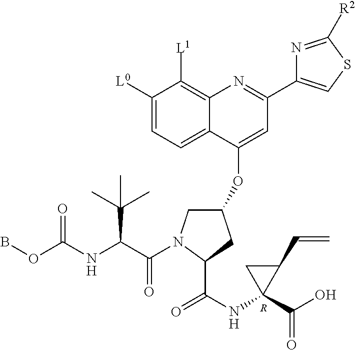

wherein B is

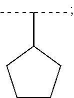

L⁰ is MeO—; L¹ is Br; and R² is

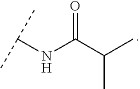

A combination therapy regimen including administering Compound (1) with an interferon-alpha and ribavirin is described in U.S. Patent Application Publication No. 2010/0068182. However, in view of the potential side-effects and overall inconvenience of treatment with an interferon (administered by injection), there is a continuing need in the field for alternative therapies for the treatment and prevention of HCV infection which do not involve the use of an interferon.

Applicants have discovered that excellent antiviral results can be achieved by combining Compound (1) with an HCV polymerase inhibitor Compound (2), as hereinafter described, and optionally ribavirin, as a combination therapy without the use of an interferon.

The following Compound (2):

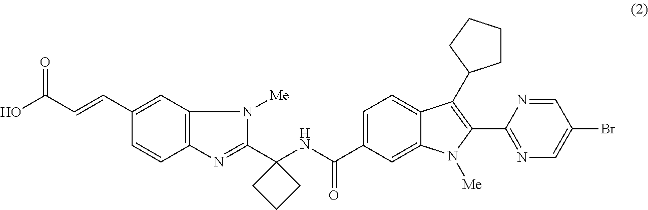

having the chemical name: (E)-3-[2-(1-{[2-(5-Bromo-pyrimidin-2-yl)-3-cyclopentyl-1-methyl-1H-indole-6-carbonyl]-amino}-cyclobutyl)-3-methyl-3H-benzimidazol-5-yl]-acrylic acid, is known as a selective and potent inhibitor of the HCV NS5B RNA-dependent RNA polymerase and useful in the treatment of HCV infection. Compound (2) falls within the scope of HCV inhibitors disclosed in U.S. Pat. Nos. 7,141,574 and 7,582,770, and US Application Publication 2009/0087409. Compound (2) is disclosed specifically as Compound #3085 in U.S. Pat. No. 7,582,770. Compound (2), and pharmaceutical formulations thereof, can be prepared according to the general procedures found in the above-cited references, all of which are herein incorporated by reference in their entirety. Preferred forms of Compound (2) include the crystalline forms, in particular the crystalline sodium salt form which is prepared as herein described.

### BRIEF SUMMARY OF THE INVENTION

The present invention provides a method of treating HCV infection or alleviating one or more symptoms thereof in a patient comprising the step of administering to the patient an effective amount of a therapeutic combination comprising Compounds (1) and (2) as herein described, or a pharmaceutically acceptable salt thereof, and optionally ribavirin. The two or three actives of the combination can be administered simultaneously or separately, as part of a regimen.

The present invention further provides for a packaged pharmaceutical composition comprising a Compound (1), which is accompanied by written instructions indicating administering Compound (1) with Compound (2) and optionally ribavirin for the treatment of HCV infection.

The present invention further provides for a packaged pharmaceutical composition comprising a Compound (2), which is accompanied by written instructions indicating administering Compound (1) with Compound (2) and optionally ribavirin for the treatment of HCV infection.

### BRIEF DESCRIPTION OF THE DRAWINGS

FIG. 1 depicts the change in HCV viral load in a group of treatment-naïve patients having chronic HCV genotype-1 infection and treated with Compound (1) sodium salt (120 mg/day), Compound (2) sodium salt (1200 mg/day) and ribavirin as combination therapy for 4 weeks, followed by combination therapy with Compound (1) sodium salt, pegylated interferon alfa-2a and ribavirin.

FIG. 2 depicts the change in HCV viral load in a group of treatment-naïve patients having chronic HCV genotype-1 infection and treated with Compound (1) sodium salt (120 mg/day), Compound (2) sodium salt (1800 mg/day) and ribavirin as combination therapy for 4 weeks, followed by combination therapy with Compound (1) sodium salt, pegylated interferon alfa-2a and ribavirin.

### DETAILED DESCRIPTION OF THE INVENTION

### Definitions

“Compound (1)” and “Compound (2)” are as defined above.

“Ribavirin” refers to 1-β-D-ribofuranosyl-1H-1,2,4-triazole-3-carboxamide, available from ICN Pharmaceuticals, Inc., Costa Mesa, Calif. and is described in the Merck Index, compound No. 8199, Eleventh Edition. Its manufacture and formulation is described in U.S. Pat. No. 4,211,771. Preferred marketed ribavirin products include REBETOL® and COPEGUS®. The term further includes derivatives or analogs thereof, such as those described in U.S. Pat. Nos. 6,063,772, 6,403,564 and 6,277,830. For example, derivatives or analogs include modified ribavirins such as 5′-amino esters, ICN Pharmaceutical's L-enantiomer of ribavirin (ICN 17261), 2′-deoxy derivatives of ribavirin and 3-carboxamidine derivatives of ribavirin, viramidine (previously known as ribamidine) and the like.

The term “pharmaceutically acceptable salt” means a salt of a Compound of formula (1) which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use.

The term includes pharmaceutically-acceptable acid addition salts and pharmaceutically-acceptable base addition salts. Lists of suitable salts are found in, e.g., S. M. Birge et al., J. Pharm. Sci., 1977, 66, pp. 1-19.

The term “pharmaceutically-acceptable acid addition salt” means those salts which retain the biological effectiveness and properties of the free bases and which are not biologically or otherwise undesirable, formed with inorganic acids such as hydrochloric acid, hydrobromic acid, sulfuric acid, sulfamic acid, nitric acid, phosphoric acid, and the like, and organic acids such as acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethane-sulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like.

The term “pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

The term “therapeutic combination” as used herein means a combination of one or more active drug substances, i.e., compounds having a therapeutic utility. Typically, each such compound in the therapeutic combinations of the present invention will be present in a pharmaceutical composition comprising that compound and a pharmaceutically acceptable carrier. The compounds in a therapeutic combination of the present invention may be administered simultaneously or separately, as part of a regimen.

### EMBODIMENTS OF THE INVENTION

According to a general embodiment, the present invention provides for a method of treating HCV infection or alleviating one or more symptoms thereof in a patient comprising the step of administering to the patient an effective amount of a therapeutic combination comprising a Compound (1) as defined herein, or a pharmaceutically acceptable salt thereof, Compound (2) as defined herein, or a pharmaceutically acceptable salt thereof, optionally together with ribavirin. An additional embodiment is directed to the use of Compound (1), or a pharmaceutically acceptable salt thereof, and Compound (2) or a pharmaceutically acceptable salt thereof, for the manufacture of pharmaceutical compositions of each compound, for use together, optionally also with ribavirin, in the treatment of HCV infection.

Additional general embodiments include a packaged pharmaceutical composition comprising a packaging containing one or more doses of Compound (1) or a pharmaceutically acceptable salt thereof, or containing one or more doses of Compound (2) or a pharmaceutically acceptable salt thereof, together with written instructions directing the co-administration of Compound (1), Compound (2) and optionally ribavirin for the treatment of HCV infection. Another embodiment is directed to a kit for the treatment of HCV infection comprising: (a) one or more doses of Compound (1) or a pharmaceutically acceptable salt thereof, and (b) one or more doses of Compound (2) or a pharmaceutically acceptable salt thereof, and (c) optionally ribavirin, together with written instructions directing the co-administration of Compound (1), Compound (2) and optionally ribavirin for the treatment of HCV infection.

In administering the therapeutic combinations of the present invention, each active agent can be administered together at the same time or separately at different times in separate dosage administrations. The present invention contemplates and includes all such dosage regimens when administering the double or triple therapeutic combinations as defined herein.

Although this combination therapy is expected to be effective against all HCV genotypes, it has been demonstrated to be particularly effective in treating HCV genotype 1 infection, including subgenotypes 1a and 1b.

The patient population to be treated with the combination therapy of the present invention can be further classified into “treatment-naïve” patients, i.e., those patient who have not received any prior treatment for HCV infection and “treatment experienced” patients, i.e, those patients who have undergone prior treatment for HCV. Either of these classes of patients may be treated with the combination therapy of the present invention. A particular class of patients that are preferably treated are those treatment experienced patients that have undergone prior interferon plus ribavirin therapy but are non-responsive to said therapy (herein “non-responders”). Such non-responders include three distinct groups of patients: (1) those who experienced <2×log₁₀ maximum reduction in HCV RNA levels during the first 12 weeks of treatment with interferon plus ribavirin (“null responders”), (2) those who experienced ≧2×log₁₀ maximum reduction in HCV RNA levels during treatment with interferon plus ribavirin but never achieve HCV RNA levels below level of detection (“partial responders”), and (3) those who achieved a virologic response with and during interferon plus ribavirin therapy but had a viral load rebound either during treatment (other than due to patient non-compliance) or after treatment has completed (“relapser”).

According to an alternative embodiment, the present invention provides a method of reducing HCV-RNA levels in a patient in need thereof, comprising the step of administering to said patient a therapeutic combination according to the present invention. Preferably, the method of the present invention reduces the HCV-RNA levels in a patient to a level below the lower limit of quantification (or “BLQ”). A BLQ level of HCV RNA as used in the present invention means a level below 25 International Units (IU) per ml of serum or plasma of a patient as measured by quantitative, multi-cycle reverse transcriptase PCR methodology according to the WHO international standard (Saladanha J, Lelie N and Heath A, Establishment of the first international standard for nucleic acid amplification technology (NAT) assays for HCV RNA. WHO Collaborative Study Group. Vox Sang 76:149-158, 1999). Such methods are well known in the art. In a preferred embodiment, the method of the present invention reduces the HCV-RNA levels in a patient to less than 25 IU per ml of serum or plasma. In another embodiment the method of the present invention reduces the HCV-RNA levels in a patient to less than a detectible level.

The usual duration of the treatment for standard interferon plus ribavirin therapy is at least 48 weeks for HCV genotype 1 infection, and at least 24 weeks for HCV genotypes 2 and 3. However, with the triple combination therapy of the present invention it may be possible to have a much shorter duration of treatment. With the triple combination therapy of the present invention the contemplated durations of treatment include at least 4 weeks, preferably at least 12 weeks, e.g., from about 12 weeks to about 24 weeks, although treatment up to and even beyond 48 weeks is possible as well. Thus, further embodiments include treatment for at least 24 weeks and for at least 48 weeks. The time period for different HCV genotypes, e.g. HCV genotypes 2, 3 or 4 is expected to be similar. Also contemplated is an initial treatment regimen with the triple combination therapy of the present invention, followed by a combination therapy of only Compound (1) with ribavirin (and with or without interferon) or followed by a combination therapy of only Compound (2) with ribavirin (and with or without interferon).

The first component of the therapeutic combination, namely, Compound (1) or a pharmaceutically acceptable salt thereof is comprised in a composition. Such a composition comprises Compound (1), or a pharmaceutically acceptable salt thereof, and a pharmaceutically acceptable adjuvant or carrier. Typical pharmaceutical compositions that may be used for Compound (1), or a pharmaceutically acceptable salt thereof, are as described in U.S. Pat. No. 7,514,557. Further specific examples of compositions are as set forth in the examples section below.

In general, the Compound (1) or a pharmaceutically acceptable salt thereof may be administered at a dosage of at least 40 mg/day (in single or divided doses). Additional embodiments for dosage amounts and ranges may include (in single or divided doses):
(a) at least 100 mg/day
(b) at least 120 mg/day
(c) at least 200 mg/day
(d) at least 240 mg/day
(e) at least 360 mg/day
(f) at least 480 mg/day
(g) from about 40 mg/day to about 480 mg/day
(h) from about 120 mg/day to about 240 mg/day
(i) from about 240 mg/day to about 480 mg/day
(j) about 120 mg/day
(k) about 240 mg/day
(l) about 360 mg/day
(m) about 480 mg/day

Although Compound (1) or a pharmaceutically acceptable salt thereof may be administered in single or divided daily doses, once a day administration (QD) of the daily dose is preferred. As the skilled artisan will appreciate, however, lower or higher doses than those recited above may be required. Specific dosage and treatment regimens for any particular patient will depend upon a variety of factors, including the age, body weight, general health status, sex, diet, time of administration, rate of excretion, drug combination, the severity and course of the infection, the patient's disposition to the infection and the judgment of the treating physician. In general, the compound is most desirably administered at a concentration level that will generally afford antivirally effective results without causing any harmful or deleterious side effects.

In another embodiment according to the invention, a loading dose amount of Compound (1) is administered for the first administration dose of the treatment. The loading dose amount is higher than the dose amount administered for subsequent administrations in the treatment. Preferably, the loading dose amount is about double in quantity, by weight, of the amount in subsequent administrations in the treatment. For example, in one embodiment, the first dose of Compound (1) administered at dosage of about 240 mg and subsequent doses of Compound (1) are administered at a dosage of about 120 mg. In another embodiment, the first dose of Compound (1) administered at a dosage of about 480 mg and subsequent doses of Compound (1) are administered at a dosage of about 240 mg. In another embodiment, the first dose of Compound (1) administered is at a dosage of about 960 mg and subsequent doses of Compound (1) are administered at a dosage of about 480 mg.

By using this loading dose concept, a clear advantage is that it is thereby possible to achieve steady state levels of active drug in the patient's system earlier than would otherwise be achieved. The blood level achieved by using a doubled loading dose is the same as would be achieved with a double dose but without the safety risk attendant to the subsequent continuous administration of a double dose. By reaching the targeted steady state level of active drug earlier in therapy also means that there less possibility of insufficient drug pressure at the beginning of therapy so that resistant viral strains have a smaller chance of emerging.

The second component of the therapeutic combination, namely, Compound (2) or a pharmaceutically acceptable salt thereof is comprised in a composition. Such a composition comprises Compound (2), or a pharmaceutically acceptable salt thereof, and a pharmaceutically acceptable adjuvant or carrier. Typical pharmaceutical compositions that may be used for Compound (1), or a pharmaceutically acceptable salt thereof, are as described in U.S. Pat. No. 7,582,770.

In general, the Compound (2) or a pharmaceutically acceptable salt thereof may be administered at dosage amounts and in dose ranges that may include (in single or divided doses):
(a) at least 800 mg/day
(b) at least 1200 mg/day
(c) at least 1800 mg/day
(d) at least 2400 mg/day
(e) from about 800 mg/day to about 2400 mg/day
(f) from about 1200 mg/day to about 1800 mg/day
(g) from about 1800 mg/day to about 2400 mg/day
(h) from about 1200 mg/day to about 2400 mg/day
(i) about 1200 mg/day
(j) about 1800 mg/day
(k) about 2400 mg/day

Although Compound (2) or a pharmaceutically acceptable salt thereof may be administered in single or divided daily doses, thrice a day administration (TID) of the divided daily dose is preferred. As the skilled artisan will appreciate, however, lower or higher doses than those recited above may be required. Specific dosage and treatment regimens for any particular patient will depend upon a variety of factors, including the age, body weight, general health status, sex, diet, time of administration, rate of excretion, drug combination, the severity and course of the infection, the patient's disposition to the infection and the judgment of the treating physician. In general, the compound is most desirably administered at a concentration level that will generally afford antivirally effective results without causing any harmful or deleterious side effects.

In another embodiment according to the invention, an induction dose amount of Compound (2) is administered for the first administration dose of the treatment. The induction dose amount is higher than the dose amount administered for subsequent administrations in the treatment. Preferably, the induction dose amount is about double to triple in quantity, by weight, of the amount in subsequent administrations in the treatment. For example, in one embodiment, the first dose of Compound (2) administered at dosage of about 1200 mg and subsequent doses of Compound (2) are administered at a dosage of about 600 mg. In another embodiment, the first dose of Compound (2) administered at a dosage of about 1200 mg and subsequent doses of Compound (2) are administered at a dosage of about 400 mg.

By using this induction dose concept, a clear advantage is that it is thereby possible to achieve a greater drop in initial viral load. Maximizing initial viral response with the first dose and then sustaining the drop with a subsequent lower dose also restricts the selection of potential resistant variants.

The optional third component of the therapeutic combination, namely ribavirin, is comprised in a pharmaceutical composition. Typically, such compositions comprise ribavirin and a pharmaceutically acceptable adjuvant or carrier and are well known in the art, including in a number of marketed ribavirin formulations. Formulations comprising ribavirin are also disclosed, e.g., in U.S. Pat. No. 4,211,771.

The types of ribavirin that may be used in the combination are as outlined hereinabove in the definitions section. In one preferred embodiment, the ribavirin is either REBETOL® or COPEGUS® and they may be administered at their labeled dosage levels indicated for interferon plus ribavirin combination therapy for the treatment of HCV infection. Of course, with the triple combination therapy of the present invention it may be possible to use a lower dosage of ribavirin, e.g., lower than is used the current standard interferon plus ribavirin therapy, while delivering the same or better efficacy than the current standard therapy with less side-effects usually associated with such therapy.

According to various embodiments, the ribavirin may be administered at dosages of (in single or divided doses):
(a) between 400 mg/day to about 1200 mg/day;
(b) between about 800 mg/day to about 1200 mg/day;
(c) between about 1000 mg/day to about 1200 mg/day;
(d) about 1000 mg/day
(e) about 1200 mg/day
(f) between about 300 mg/day to about 800 mg/day
(g) between about 300 mg/day to about 700 mg/day
(h) between 500 mg/day to about 700 mg/day
(i) between 400 mg/day to about 600 mg/day
(j) about 400 mg/day
(k) about 600 mg/day
(l) about 800 mg/day

According to one embodiment, the ribavirin composition comprises ribavirin in a formulation suitable for dosing once a day, twice daily, thrice daily, four times a day, five times a day, or six times a day. For example, if a therapeutic combination comprises about 1000 mg/day dosage of ribavirin, and a dosing of five times a day is desired, then the therapeutic combination will comprise ribavirin in a formulation, e.g., a tablet, containing, e.g., about 200 mg of ribavirin.

For example, in one embodiment the present invention contemplates a method of treating hepatitis C viral (HCV) infection or alleviating one or more symptoms thereof in a patient comprising the step of administering to the patient a therapeutic combination comprising:
(a) Compound (1) or a pharmaceutically acceptable salt thereof at a dosage between about 48 mg per day and about 480 mg per day;
(b) Compound (2) or a pharmaceutically acceptable salt thereof at a dosage between about 800 mg/day to about 2400 mg/day; and
(c) optionally ribavirin at a dosage of between about 400 mg/day to about 1200 mg/day.

In another embodiment the present invention contemplates a method of treating hepatitis C viral (HCV) infection or alleviating one or more symptoms thereof in a patient comprising the step of administering to the patient a therapeutic combination comprising:
(a) Compound (1) or a pharmaceutically acceptable salt thereof at a dosage between about 120 mg/day to about 240 mg/day;
(b) Compound (2) or a pharmaceutically acceptable salt thereof at a dosage between about 1200 mg/day to about 1800 mg/day; and
(c) optionally ribavirin at a dosage of between about 1000 mg/day to about 1200 mg/day.

In another embodiment the present invention contemplates a method of treating hepatitis C viral (HCV) infection or alleviating one or more symptoms thereof in a patient comprising the step of administering to the patient a therapeutic combination comprising:
(a) Compound (1) or a pharmaceutically acceptable salt thereof at a dosage of about 120 mg/day;
(b) Compound (2) or a pharmaceutically acceptable salt thereof at a dosage of about 1200 mg/day or about 1800 mg/day; and
(c) optionally ribavirin at a dosage of between about 1000 mg/day to about 1200 mg/day.

Further embodiments include any of the above-mentioned embodiments, and where:
(a) the therapy is a triple combination therapy including administration of Compound (1) or a pharmaceutically acceptable salt thereof, Compound (2) or a pharmaceutically acceptable salt thereof and ribavirin; or
(b) the therapy is a double combination therapy including administration of Compound (1) or a pharmaceutically acceptable salt thereof and Compound (2) or a pharmaceutically acceptable salt thereof, i.e., without any additional anti-HCV agents.

Further embodiments include any of the above-mentioned embodiments, and where:
(a) the HCV infection is genotype 1 and the patient is a treatment-naïve patient; or
(b) the HCV infection is genotype 1 and the patient is a treatment-experienced patient who is non-responsive to a combination therapy of interferon plus ribavirin.

Further embodiments include any of the above-mentioned embodiments, and where the Compound (1) or a pharmaceutically acceptable salt thereof is administered once a day, the Compound (2) or a pharmaceutically acceptable salt thereof is administered three times a day and the ribavirin; if included in the therapy, is administered twice a day.

Further embodiments include any of the above-mentioned embodiments and where the loading dose concept in used for Compound (1), e.g., the first dose of Compound (1) administered is double in quantity to the subsequent doses.

Further embodiments include any of the above-mentioned embodiments, and where the therapeutic regimen of the present invention is administered to the patient for at least about 4 weeks, more preferably at least about 12 weeks, at least about 16 weeks, at least about 24 weeks, at least about 28 weeks or at least about 40 weeks.

With respect to the double or triple combination therapies of the present invention, the present invention contemplates and includes all combinations of the various preferred embodiments and sub-embodiments as set forth herein.

An additional embodiment is directed to a packaged pharmaceutical composition comprising a packaging containing one or more doses of Compound (1) or a pharmaceutically acceptable salt thereof, or containing one or more doses of Compound (2) or a pharmaceutically acceptable salt thereof, each together with written instructions directing the co-administration of Compound (1), Compound (2) and optionally ribavirin for the treatment of HCV infection. In another embodiment, one or more doses of Compound (1), or a pharmaceutically acceptable salt thereof, and one or more doses of Compound (2), or a pharmaceutically acceptable salt thereof, and optionally ribiavrin, are placed together in a single packaging forming a so-called “kit”, which includes written instructions directing the co-administration of Compound (1), Compound (2) and optionally ribavirin for the treatment of HCV infection. In either case, the individual doses of Compound (1) or a pharmaceutically acceptable salt thereof, or Compound (2) or a pharmaceutically acceptable salt thereof, can be in the form of any of the standard pharmaceutical dosage forms, e.g. tablets, capsules, and packaged within any of the standard types of pharmaceutical packaging materials, e.g. bottles, blister-packs, etc., that may themselves be contained within an outer packaging material such as a paper/cardboard box. The written instructions will typically be provided either on the packaging material(s) itself or on a separate paper (a so-called “package insert”) that is provided together with the dosage forms within the outer packaging material. All such packaging embodiments and variations thereof are embraced by the present invention.

Additionally, surprising results have been seen in the excellent antiviral activity and suppression of HCV viral replication and limited emergence of viral resistance during the combination therapy treatment contemplated by the present invention. Accordingly, in an additional embodiment, there is limited or no emergence of viral resistance during the combination therapy of the present invention. In a further embodiment, there is limited or no emergence of HCV variants that encode HCV NS3 protease amino acid substitutions at one or more of R155 and/or D168 and/or A156 during the combination therapy of the present invention In a further embodiment there is limited or no emergence of HCV variants that encode HCV NS5B polymerase amino acid substitutions at P495 during the combination therapy of the present invention. In a more specific embodiment there is limited or no emergence of HCV variants that encode both HCV NS3 protease amino acid substitutions (NS3R155 and/or NS3D168 and/or NS3A156) and HCV NS5B polymerase amino acid substitutions P495 during the combination therapy of the present invention

Further embodiments include any of the above-mentioned embodiments, and where either:
(a) the HCV infection is genotype 1a and the patient is a treatment-naïve patient; or
(b) the HCV infection is genotype 1a and the patient is a treatment-experienced patient who is non-responsive to a combination therapy of interferon plus ribavirin;

and wherein there is limited or no emergence of variants that encode substitutions at NS3 protease amino acid R155 and substitutions at NS5B polymerase P495 during the combination therapy of the present invention.

### EXAMPLES

I. Methods for Preparing Compound (1)

Methods for preparing amorphous Compound (1) and a general description of pharmaceutically acceptable salt forms can be found in U.S. Pat. Nos. 6,323,180, 7,514,557 and 7,585,845. Methods for preparing additional forms of Compound (1), in particular the crystalline sodium salt form, can be found in U.S. Patent Application Publication No. 2010/0093792.

II. Formulations of Compound (1)

One example of a pharmaceutical formulation of Compound (1) include an oral solution formulation as disclosed in WO 2010/059667. Additional examples include capsules containing a lipid-based liquid formulation, as disclosed in WO 2011/005646. Examples of such capsule formulations are described below.

### Example 1

### Softgel Capsule Formulation #1

The composition of the liquid fill formulation:

| Ingredient | Monograph | Functionality | % w/w |
|---|---|---|---|
| Compound (1) Na salt | | API | 15.0 |
| Mono-, Diglycerides of | | Lipid | 46.3 |
| Caprylic/Capric Acid | | | |
| (Capmul ® MCM) | | | |
| Polyoxyl 35 Castor Oil | NF | Surfactant | 30.8 |
| (Cremophor ® EL) | | | |
| Propylene Glycol | USP | Solvent | 7.7 |
| DL-α-tocopherol | USP | Anti-oxidant | 0.2 |
| Total | | | 100.0 |

Two specific soft-gel capsule drug product formulations were prepared according to the above general Formulation #1, a 40 mg product and a 120 mg product:

| | | 40 mg | 120 mg |
|---|---|---|---|
| Ingredient | Function | mg/capsule | mg/capsule |
| Compound (1) Na salt | Drug | 42.30¹ | 126.90² |
| (milled) | substance | | |
| Mono/Diglycerides of | Lipid phase | 130.57 | 391.70 |
| Caprylic/Capric Acid | | | |
| Polyoxyl 35 Castor Oil (NF) | Surfactant | 86.86 | 260.57 |
| Macrogolglycerol | | | |
| Ricinoleate (Ph. Eur.) | | | |
| Propylene Glycol | Solvent | 21.71 | 65.14 |
| Vitamin E (dl-alpha | Anti- | 0.56 | 1.69 |
| tocopherol) (USP) | oxidant | | |
| All-rac-alpha-tocopherol | | | |
| (Ph. Eur.) | | | |
| Nitrogen³ | Processing | q.s. | q.s. |
| | aid | | |
| Total Fill Weight | | 282.00 | 846.00 |
| Soft Gelatin Capsule | Shell | 280⁴ | 590⁵ |
| Shell | | | |
| Wet Total Capsule | | 562 | 1436 |
| Weight | | | |
| Dry Total Capsule | | 480 | 1250 |
| Weight | | | |

¹42.30 mg of Compound (1) Na salt is equivalent to 40.0 mg of the active moiety.
²126.90 mg of Compound (1) Na salt is equivalent to 120.0 mg of the active moiety.
³Nitrogen is used as a processing aid and does not appear in the final product.
⁴The approximate weight of the capsule shell before drying and finishing is 280 mg. The approximate weight of the capsule shell after drying and finishing is 198 mg.
⁵The approximate weight of the capsule shell before drying and finishing is 590 mg. The approximate weight of the capsule shell after drying and finishing is 404 mg.

### Example 2

### Softgel Caspule Formulation #2

The composition of the liquid fill formulation:

| Ingredient | Monograph | Functionality | % w/w |
|---|---|---|---|
| Compound (1) Na salt | | API | 15.0 |
| Mono-, Diglycerides of | | Lipid | 42.4 |
| Caprylic/Capric Acid | | | |
| (Capmul ® MCM) | | | |
| Polyoxyl 35 Castor Oil | NF | Surfactant | 33.9 |
| (Cremophor ® EL) | | | |
| Propylene Glycol | USP | Solvent | — |
| Oleic Acid | | Lipid | 8.5 |
| DL-α-tocopherol | USP | Anti-oxidant | 0.2 |
| Total | | | 100.0 |

A specific 150 mg soft-gel capsule drug product formulation was prepared according to the above general formula.

### Example 3

### Hard Shell Capsule Formulation #3

The composition of the liquid fill formulation:

| Ingredient | Monograph | Functionality | % w/w |
|---|---|---|---|
| Compound (1) Na salt | | API | 20.0 |
| Mono-, Diglycerides of | | Lipid | 53.8 |
| Caprylic/Capric Acid | | | |
| (Capmul ® MCM) | | | |
| Polyoxyl 35 Castor Oil | NF | Surfactant | 23.0 |
| (Cremophor ® EL) | | | |
| Propylene Glycol | USP | Solvent | 3.0 |
| DL-α-tocopherol | USP | Anti-oxidant | 0.2 |
| Total | | | 100.0 |

A specific 150 mg hard-shell capsule drug product formulation was prepared according to the above general formula.

Preparation of Formulations 1-3:

The drug substance is jet-milled to remove large aggregates so that the mixing time for the bulk fill manufacturing will be consistent and reasonably short. The target particle size distribution of the drug substance is to reduce the x90 (v/v) to no more than 10 micron and the x98 (v/v) to no more than 20 micron as measured by Sympatec. All the excipients in the fill formulation are combined in a mixing vessel and mixed until uniform prior to adding the drug substance. After addition of the drug substance, mixing continues until the fill solution is clear by visual inspection. A nitrogen blanket over the fill solution is used throughout the preparation as a standard practice. The fill solution is passed through a filter to remove any extraneous particles. Encapsulation of the filtered bulk fill material in capsules is performed utilizing standard soft gelatin or hard gelatin capsule technology and in-process controls. Filled capsules are dried and then washed with a finishing/wash solution prior to packaging resulting in shiny, pharmaceutically elegant capsules.

III. Methods for Preparing Compound (2)

Methods for preparing amorphous Compound (2) can be found in U.S. Pat. Nos. 7,141,574 and 7,582,770, and US Application Publication 2009/0087409.

The following Example provides the method for preparing an additional form of Compound (2), the sodium salt form, that may be used in the present invention.

### Example 4

### Preparation of Compound (2) Sodium Salt

### Step 1. Synthesis of Isopropyl 3-Cyclopentyl-1-methyl-1H-indole-6-carboxylate

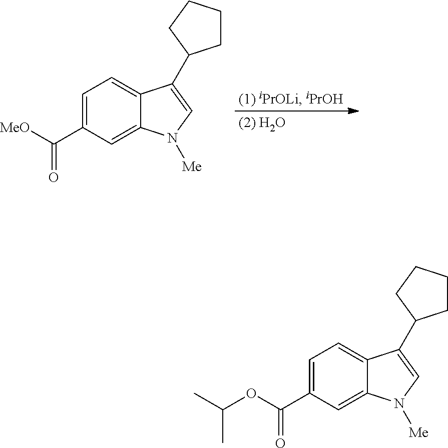

Because of the instability of brominated product, methyl 3-cyclopentyl-1-methyl-1H-indole-6-carboxylate needed to be converted into the more stable isopropyl 3-cyclopentyl-1-methyl-1H-indole-6-carboxylate via a simple and high yielding operation. The conversion worked the best with stoichiometric amounts of solid lithium isopropoxide. Use of 0.1 eq lithium isopropoxide led to longer reaction times and as a result to more hydrolysis by-product, while lithium isopropoxide solution in THF caused a problematic isolation and required distillation of THF.

Procedure:

The mixture of methyl 3-cyclopentyl-1-methyl-1H-indole-6-carboxylate (50.0 g, 0.194 mol) and lithium isopropoxide (16.2 g, 95%, 0.233 mol) in 2-propanol was stirred at 65±5° C. for at least 30 min for complete trans-esterification. The batch was cooled to 40±5° C. and water (600 g) was added at a rate to maintain the batch temperature at 40±5° C. After addition, the mixture was cooled to 20-25° C. over 2±0.5 h and held at 20-25° C. for at least 1 h. The batch was filtered and rinsed with 28 wt % 2-propanol in water (186 g), and water (500 g). The wet cake was dried in vacuo (≦200 Torr) at 40-45° C. until the water content was ≦0.5% to give isopropyl 3-cyclopentyl-1-methyl-1H-indole-6-carboxylate (52.7 g, 95% yield) in 99.2 A % (240 nm).

The starting material methyl 3-cyclopentyl-1-methyl-1H-indole-6-carboxylate can be prepared as described in Example 12 of U.S. Pat. No. 7,141,574, and in Example 12 of U.S. Pat. No. 7,642,352, both herein incorporated by reference.

### Step 2. Synthesis of Isopropyl 2-Bromo-3-cyclopentyl-1-methyl-1H-indole-6-carboxylate

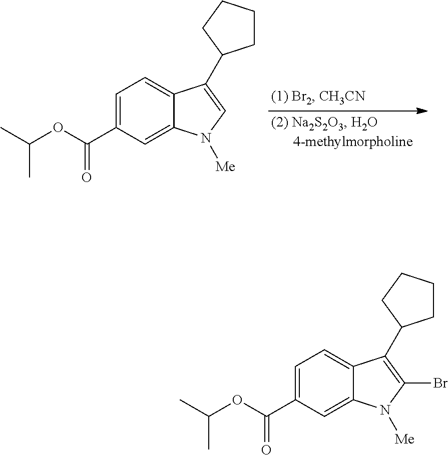

This process identified the optimal conditions for the synthesis of 2-bromo-3-cyclopentyl-1-methyl-1H-indole-6-carboxylate via bromination of the corresponding 3-cyclopentyl-1-methyl-1H-indole-6-carboxylate with bromine. It's very important to control the reaction temperature and to quench the reaction mixture with a mixture of aqueous sodium thiosulfate and 4-methylmorpholine to minimize the formation of the dibromo- and 2-indolone impurities. Further neutralization of the crude product with NaOH in isopropanol greatly increases the stability of the isolated product.

Procedure:

The mixture of isopropyl 3-cyclopentyl-1-methyl-1H-indole-6-carboxylate (50.0 g, 0.175 mol) and acetonitrile (393 g) was cooled to −6±3° C. Bromine (33.6 g, 0.210 mol) was added while the batch was maintained at −6±3° C. The resulting slurry was stirred at −6±3° C. for at least 30 min. When HPLC showed ≧94% conversion (the HPLC sample must be quenched immediately with aqueous 4-methylmorpholine/sodium thiosulfate solution), the mixture was quenched with a solution of sodium thiosulfate (15.3 g) and 28.4 g 4-methylmorpholine in water (440 g) while the temperature was maintained at −5±5° C. After it was stirred at 0±5° C. for at least 2 h, the batch was filtered and rinsed with 85 wt % methanol/water solution (415 g), followed by water (500 g), and dried until water content is ≦30%. The wet cake was suspended in 2-propanol (675 g), and heated to 75±5° C. The resulting hazy solution was treated with 1.0 M aqueous sodium hydroxide solution (9.1 g) and then with 135.0 g water at a rate to maintain the batch at 75±5° C. The suspension was stirred at 75±5° C. for at least 30 min, cooled to 15±2° C. over 30-40 min, and held at 15±2° C. for at least 1 h. The batch was filtered, rinsed with 75 wt % 2-propanol/water solution (161 g), and dried in vacuo (≦200 Torr) at 50-60° C. until the water content was ≦0.4% to give isopropyl 2-bromo-3-cyclopentyl-1-methyl-1H-indole-6-carboxylate as a solid (55.6 g, 87% yield) in 99.5 A % (240 nm) and 97.9 Wt %.

Alternative Procedure:

The mixture of isopropyl 3-cyclopentyl-1-methyl-1H-indole-6-carboxylate (84 g, 0.294 mol) and isopropyl acetate (1074 g) was cooled to between −10-0° C. Bromine (50 g, 0.312 mol) was added while the batch was maintained at −10-0° C. The resulting slurry was stirred at the same temperature for additional 30 min and quenched with a pre-cooled solution of sodium thiosulfate pentahydrate (13 g) and triethylamine (64.5 g) in water (240 g) while the temperature was maintained at 0-10° C. The mixture was heated to 40-50° C. and charged with methanol (664 g). After it was stirred at the same temperature for at least 0.5 h, the batch was cooled to 0-10° C. and stirred for another 1 hr. The precipitate was filtered, rinsed with 56 wt % methanol/water solution (322 g), and dried in vacuo (≦200 Torr) at 50-60° C. until the water content was ≦0.4% to give isopropyl 2-bromo-3-cyclopentyl-1-methyl-1H-indole-6-carboxylate as a beige solid (90-95 g, 80-85% yield).

### Step 3a,b. Preparation of Compound I by One-Pot Pd-Catalyzed Borylation-Suzuki Coupling Reaction

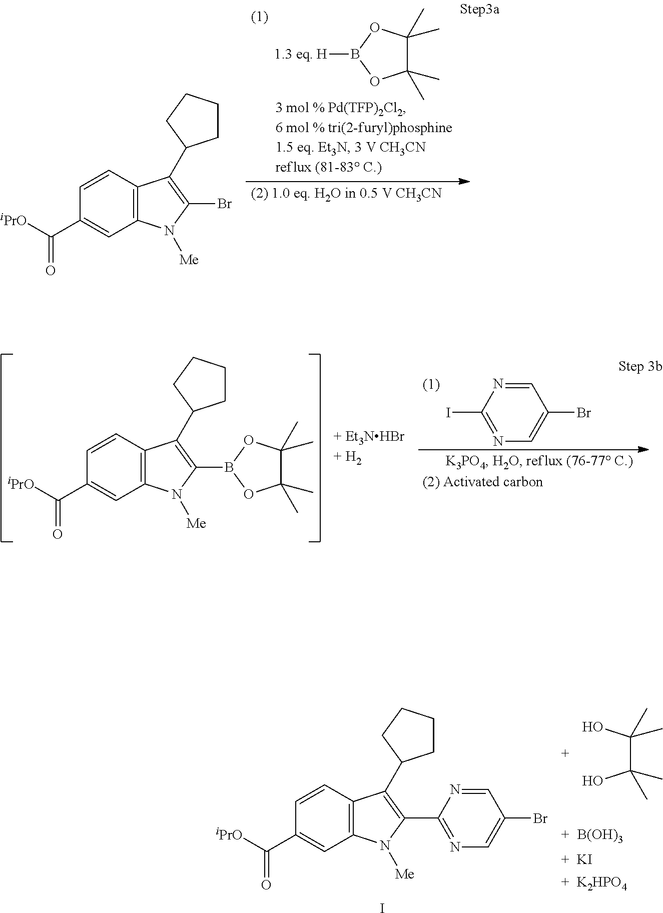

To a clean and dry reactor containing 20.04 g of isopropyl 2-bromo-3-cyclopentyl-1-methyl-1H-indole-6-carboxylate, 1.06 g of Pd(TFP)₂Cl₂ (3 mol %) and 0.76 g of tri(2-furyl)phosphine (6 mol %) was charged 8.35 g of triethylamine (1.5 equivalent), 39.38 g of CH₃CN at 23±10° C. under nitrogen or argon and started agitation for 10 min. 9.24 g of 4,4,5,5-tetramethyl-1,3,2-dioxaborolane was charged into the reactor. The mixture was heated to reflux (ca. 81-83° C.) and stirred for 6 h until the reaction completed. The batch was cooled to 30±5° C. and quenched with a mixture of 0.99 g of water in 7.86 g of CH₃CN. 17.24 g of 5-bromo-2-iodopyrimidine and 166.7 g of degassed aqueous potassium phosphate solution (pre-prepared from 46.70 g of K₃PO₄ and 120 g of H₂O) was charged subsquently under argon or nitrogen. The content was heated to reflux (ca. 76-77° C.) for 2 h until the reaction completed. 4.5 g of 1-methylimidazole was charged into the reactor at 70° C. The batch was cooled to 20±3° C. over 0.5 h and hold at 20±3° C. for at least 1 h. The solid was collected by filtration. The wet cake was first rinsed with 62.8 g of 2-propanol, followed by 200 g of H₂O. The solid was dried under vacuum at the temperature below 50° C.

Into a dry and clean reactor was charged dried I, 10 wt % Norit SX Ultra and 5 V of THF. The content was heated at 60±5° C. for at least 1 h. After the content was cooled to 35±5° C., the carbon was filtered off and rinsed with 3 V of THF. The filtrate was charged into a clean reactor containing 1-methylimidazole (10 wt % relative to I). After removal of 5 V of THF by distillation, the content was then cooled to 31±2° C. After the agitation rate was adjusted to over 120 rpm, 2.5 V of water was charged over a period of at least 40 minutes while maintaining the content temperature at 31±2° C. After the content was agitated at 31±2° C. for additional 20 min, 9.5 V of water was charged into the reactor over a period of at least 30 minutes at 31±2° C. The batch was then cooled to about 25±3° C. and stirred for additional 30 minutes. The solid was collected and rinsed with 3 V of water. The wet product I was dried under vacuum at the temperature below 50° C. (19.5 g, 95 wt %, 76% yield).

Alternative Procedure:

To a clean and dry reactor containing 40 g of isopropyl 2-bromo-3-cyclopentyl-1-methyl-1H-indole-6-carboxylate (0.110 mol), 0.74 g of Pd(OAc)₂ (3.30 mmol, 3 mol % equiv.) and 3.2 g of tri(2-furyl)phosphine (13.78 mmol, 12.5 mol % equiv.) was charged 16.8 g of triethylamine (1.5 equivalent), 100 mL of acetonitrile at 25° C. under nitrogen or argon. 20.8 g of 4,4,5,5-tetramethyl-1,3,2-dioxaborolane was charged into the reactor within 30 min. The mixture was heated to reflux (ca. 81-83° C.) and stirred for over 5 hrs until the reaction completed. The batch was cooled to 20° C. and quenched with a mixture of 2.7 g of water in 50 mL of CH₃CN. The batch was warmed to 30° C., stirred for 1 hr and transferred to a second reactor containing 34.4 g of 5-bromo-2-iodopyrimidine in 100 mL of acetonitrile. The reactor was rinsed with 90 mL of acetonitrile. To the second reactor was charged with degassed aqueous potassium phosphate solution (pre-prepared from 93.2 g of K₃PO₄ and 100 g of H₂O) under argon or nitrogen. The content was heated to reflux (ca. 80° C.) for over 3 h until the reaction completed. 9.2 g of 1-methylimidazole was charged into the reactor at 70° C. and the mixture was stirred for at least 10 min. The aqueous phase was removed after phase separation. 257 g of isopropanol was charged at 70° C. The batch was cooled slowly to 0° C. and hold for at least 1 h. The solid was collected by filtration. The wet cake was rinsed twice with 2-propanol (2×164 g) and dried under vacuum at the temperature below 50° C. to give I as a yellow to brown solid (26 g, 75% yield).

### Step 4. Hydrolysis of I to II

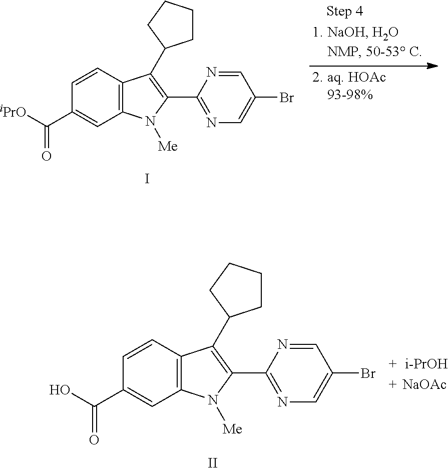

I (20 g) and 1-methyl-2-pyrrolidinone (NMP) (113 g) were charged into a clean reactor under nitrogen. After the batch was heated to 50-53° C. with agitation, premixed aq. NaOH (5.4 g of 50% aq. NaOH and 14.3 g of water) was introduced into the reactor. The resulting mixture was stirred at 50-53° C. for about 10 hrs until the reaction completed. A premixed aq. HOAc (60 g of water and 9.0 g of HOAc) was added over 0.5 h at 45±5 to reach pH 5.5-7.5. The batch was cooled to 20±5° C. and then kept for at least 1.0 h. The solid product was collected and rinsed with 80 g of NMP/water (1:3 volume ratio) and then 60 g of water. The product was dried under vacuum at the temperature below 50° C. to give II as a pale yellow powder (19-20 g, purity >99.0 A % and 88.4 wt %, containing 5.4 wt % NMP). The yield is about 93-98%.

Notes: The original procedure used for the hydrolysis of I was carried out with aq. NaOH (2.5 eq) in MeOH/THF at 60° C. Although it has been applied to the preparation of II on several hundred grams scale, one disadvantage of this method is the formation of 5-MeO pyrimidine during hydrolysis (ca. 0.4 A %), which is extremely difficult to remove in the subsequent steps. In addition, careful control has to be exerted during crystallization. Otherwise, a thick slurry might form during acidification with HOAc. The use of NMP as solvent could overcome all aforementioned issues and give the product with desired purity.

Alternative Process

To a reactor was charged I (71 g), isopropanol (332 g), aqueous NaOH (22 g, 45 wt %) and water (140 g) at ambient temperature. The mixture was heated to reflux (80° C.) and stirred for at least 3 hrs until the reaction completed. The batch was cooled to 70° C. and charged a suspension of charcoal (3.7 g) in isopropanol (31 g). The mixture was stirred at the same temperature for over 10 min and filtered. The residue was rinsed with isopropanol (154 g). Water (40 g) was charged to the filtrate at 70-80° C., followed by slow addition of 36% HCl solution (20 g) to reach pH 5-6. The batch was stirred for over 30 min at 70° C., then cooled to 20° C. over 1 hr and kept for at least 1.0 h. The solid product was collected and rinsed with 407 g of isopropanol/water (229 g IPA, 178 g H₂O). The product was dried under vacuum at 80° C. for over 5 hrs to give II as a white powder (61 g, 95% yield).

Notes on Steps 5 to 8 Below:

A concise and scalable 4-step process for the preparation of the benzimidazole intermediate V was developed. The first step was the preparation of 4-chloro-2-(methyl)-aminonitrobenzene starting from 2,4-dichloronitrobenzene using aqueous methyl amine in DMSO at 65° C. Then, a ligandless Heck reaction with n-butyl acrylate in the presence of Pd(OAc)₂, ⁱPr₂NEt, LiCl, and DMAc at 110° C. was discovered.

### Step 5: SNAr Reaction of (5-chloro-2-nitrophenyl)-methylamine

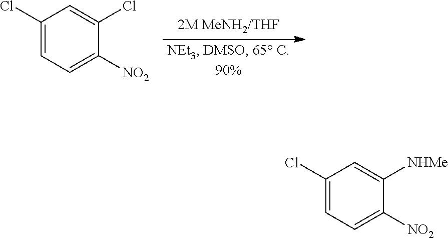

To a solution of (5-chloro-2-nitrophenyl)-methylamine (40 g, 208.3 mmol, 1 equiv) in DMSO (160 mL) was added 40% MeNH₂ solution in water (100 mL, 1145.6 mmol, 5.5 eq) slowly keeping the temperature below 35° C. The reaction was stirred at r.t. until the complete consumption of the starting material (>10 h). Water (400 mL) was added to the resulting orange slurry and stirred at r.t. for additional 2 h. The solid was filtered, rinsed with water (200 mL) and dried under reduced pressure at 40° C. (5-chloro-2-nitrophenyl)-methylamine (36.2 g, 93% yield, 94 A % purity) was isolated as a solid.

### Step 6: Heck Reaction of (5-chloro-2-nitrophenyl)-methylamine

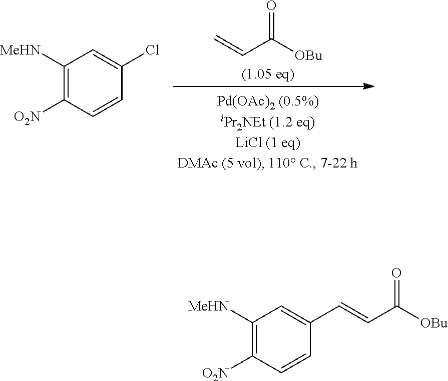

To a mixture of 4-chloro-2-methylaminonitrobenzene (50.0 g, 268.0 mmol, 1.0 eq), Pd(OAc)₂ (0.30 g, 1.3 mmol, 0.005 eq) and LiCl (11.4 g 268.0 mmol, 1.0 eq) in DMAc (250 mL) was added ⁱPr₂NEt (56 mL, 321.5 mmol, 1.2 eq) followed by n-butyl acrylate (40 mL, 281.4 mmol, 1.05 eq) under nitrogen. The reaction mixture was stirred at 110° C. for 12 h, then cooled to 50° C. 1-methylimidazole (10.6 mL, 134.0 mmol, 0.5 eq) was added and the mixture was stirred for 30 min before filtering and adding water (250 mL). The resulting mixture was cooled to r.t. over 1 h. The resulting solid was filtered and washed with water and dried to yield n-butyl 3-methylamino-4-nitrocinnamate (71.8 g, 96%, 99.2 A % purity).

### Step 7: Reduction of n-butyl (3-methylamino-4-nitro)-cinnamate

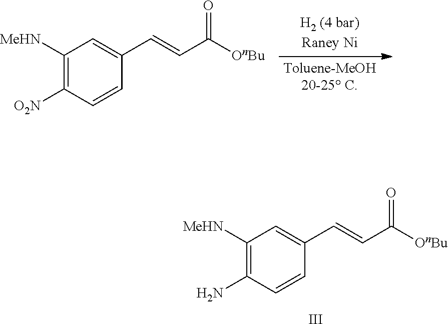

To a reactor was charged n-butyl 3-methylamino-4-nitrocinnamate (70.0 g, mmol, 1.0 eq), Raney Ni (4.9 g, ˜20 wt % H₂O), charcoal “Norit SX Ultra” (3.5 g), toluene (476 mL) and MeOH (224 mL). The reactor was charged with hydrogen (4 bar) and the mixture was stirred at 20-25° C. for about 2 hrs until the reaction was completed. The reaction mixture was filtered and rinsed the filter residue with toluene (70 mL). To the combined filtrates were added “Norit SX Ultra” charcoal (3.5 g). The mixture was stirred at 50° C. for 1.0 hr and filtered. The filtrate was concentrated under reduced pressure to remove solvents to 50% of the original volume. The remained content was heated to 70° C. and charged slowly methyl cyclohexane (335 mL) at the same temperature. The mixture was cooled to about 30-40° C. and seeded with III seed crystals, then slowly cooled the suspension to ˜−10° C. The solid was filtered and rinsed with methyl cyclohexane in three portions (3×46 mL). The wet cake was dried in vacuo at 40° C. to give III (53.3 g, 215 mmol, 86%).

### Step 8: Preparation of Benzimidazole V

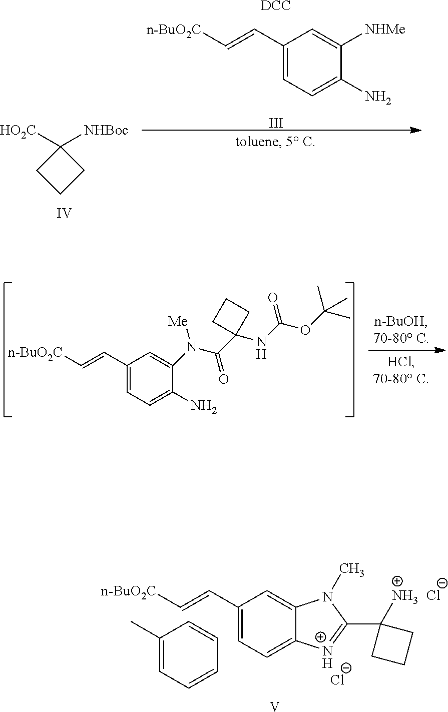

To reactor-1 was charged III (35 g, 140.95 mmol) in toluene (140 g). The mixture was heated to 50° C. to obtain a clear solution. To a second reactor was charged IV (36.4 g, 169.10 mmol) and toluene (300 g), followed by addition of a solution of dicyclohexyl carbodimide (11.6 g, in 50% toluene, 28.11 mmol) at 0-10° C. The mixture was stirred at the same temperature for 15 min, then charged parallelly with the content of reactor-1 and the solution of dicyclohexyl carbodimide (52.4 g, in 50% toluene, 126.98 mmol) within 1 hr while maintaining the batch temperature at 0-10° C. The mixture was agitated at the same temperature for 3 hrs, and warmed to 25° C. for another 1 hr. Once III was consumed, toluene (˜300 mL) was distilled off under reduced pressure at 70-80° C. n-Butanol (200 g) was added, followed by 3 M HCl solution in n-butanol (188 g) while maintaining the temperature at 70-80° C. (Gas evolution, product precipitates). After stirring for over 30 min. at 70-80° C., the mixture was cooled to 20-30° C. over 1 hr. The precipitate was filtered and washed with acetone (172 g) and toluene (88 g). The wet cake was dried in vacuo at ˜60° C. to give V toluene solvate as off white solid (60-72 g, 85-95% yield). Compound V could be used directly for the next step or basified prior to next step to obtain the free base compound VI used in the next step.

### Step 9. Synthesis of (E)-Butyl 3-(2-(1-(2-(5-Bromopyrimidin-2-yl)-3-cyclopentyl-1-hydroxy-1H-indole-6-carboxamido)cyclobutyl)-1-methyl-1H-benzo[d]imidazol-6-yl)acrylate VII

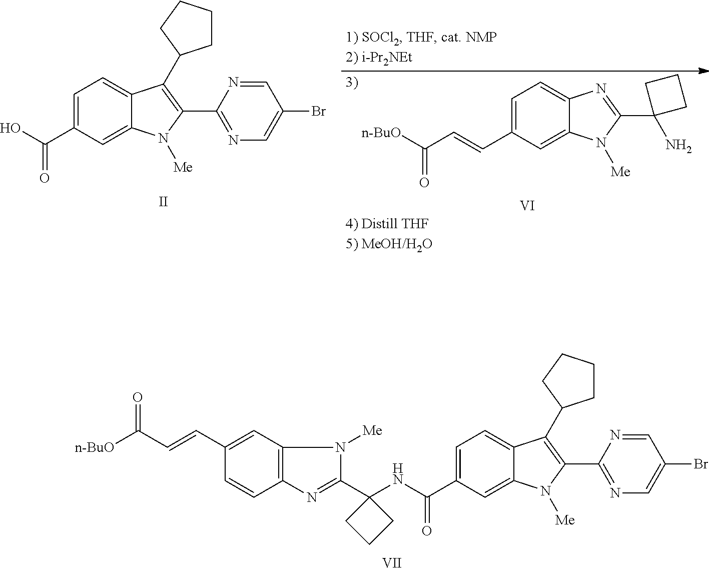

Notes:

The conversion of the acid into acid chloride was achieved using inexpensive thionyl chloride in the presence of catalytic amount of NMP or DMF. An efficient crystallization was developed for the isolation of the desired product in high yield and purity.

Procedure (Using Free Base VI):

To the suspension of 2-(5-bromopyrimidin-2-yl)-3-cyclopentyl-1-methyl-1H-indole-6-carboxylic acid II (see Step 4) (33.36 g, 90.0 wt %, containing ˜0.2 equiv of NMP from previous step, 75.00 mmol) in THF (133.4 g) was added thionyl chloride (10.71 g). The mixture was stirred at 25±5° C. for at least 1 h. After the conversion was completed as determined by HPLC (as derivative of diethylamine), the mixture was cooled to 10±5° C. and N,N-diisopropylethylamine (378.77 g, 300 mmol) below 25° C. A solution of (E)-butyl 3-(2-(1-aminocyclobutyl)-1-methyl-1H-benzo[d]imidazol-6-yl)acrylate VI (25.86 g, 97.8 Wt %, 77.25 mmol) dissolved in THF (106.7 g) was added at a rate to maintain the temperature of the content ≦25° C. The mixture was stirred at 25±5° C. for at least 30 min for completion of the amide formation. The mixture was distilled at normal pressure to remove ca.197 mL (171.5 g) of volatiles (Note: the distillation can also be done under reduced pressure). The batch was adjusted to 40±5° C., and MeOH (118.6 g) was added. Water (15.0 g) was added and the mixture was stirred at 40±5° C. until crystallization occurred (typically in 30 min), and held for another 1 h. Water (90 g) was charged at 40±5° C. over 1 h, and the batch was cooled to 25±5° C. in 0.5 h, and held for at least 1 h. The solid was filtered, rinsed with a mixture of MeOH (39.5 g), water (100 g), and dried in vacuo 200 Torr) at 50±5° C. to give (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate VII (51.82 g, 96.6% yield) with a HPLC purity of 98.0 A % (240 nm) and 99.0 Wt %.

Alternative Process (Using Compound V from Step 8)

To reactor 1 was charged 2-(5-bromopyrimidin-2-yl)-3-cyclopentyl-1-methyl-1H-indole-6-carboxylic acid II (33.6 g), toluene (214 g) and N-methylpyrrolidone (1.37 g). The mixture was heated to 40° C., then added a solution of thionyl chloride (13 g) in toluene (17 g). The mixture was stirred at 40° C. for at least 0.5 h and cooled to 30° C. To a second reactor was charged with compound V (the bis-HCl salt toluene solvate from Step 8) (39.4 g), toluene (206 g) and N,N-diisopropylethylamine (70.8 g) at 25° C. The content of reactor 1 was transferred to reactor 2 at 30° C. and rinsed with toluene (50 g). The mixture was stirred at 30° C. for another 0.5 h, then charged with isopropanol (84 g) and water (108 g) while maintained the temperature at 25° C. After stirring for 10 min, remove the aqueous phase after phase cutting. To the organic phase was charged isopropanol (43 g), water (54 g) and stirred for 10 min. The aqueous phase was removed after phase cutting. The mixture was distilled under reduced pressure to remove ca.250 mL of volatiles, followed by addition of methyl tert-butyl ether (MTBE, 238 g). The batch was stirred at 65° C. for over 1 hr, then cooled to 20 C over 1 hr and held for another 1 hr at the same temperature. The solid was filtered, rinsed with MTBE (95 g), and dried in vacuo at 80° C. to give (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate VII as a beige solid (50 g, 90% yield).

### Step 10. Synthesis of (E)-3-(2-(1-(2-(5-Bromopyrimidin-2-yl)-3-cyclopentyl-1-methyl-1H-indole-6-carboxamido)cyclobutyl)-1-methyl-1H-benzo[d]imidazol-6-yl)acrylic acid (Compound (1))

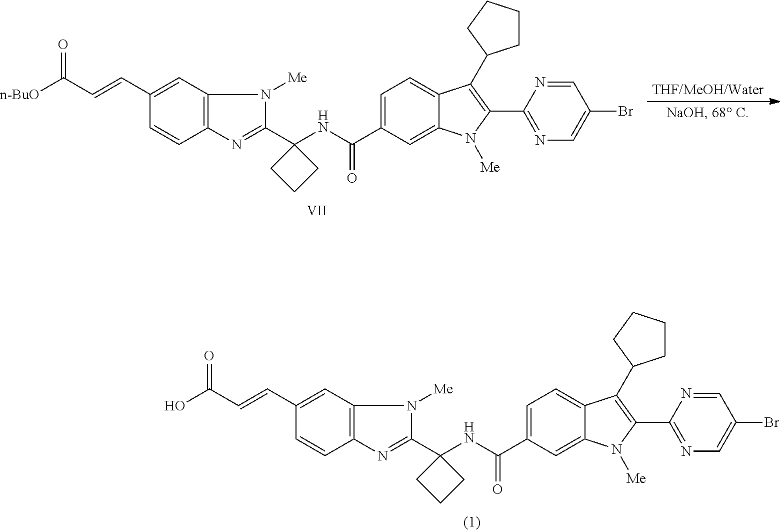

Notes:

In this process, hydrolysis of (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate was carried out in mixture of THF/MeOH and aq NaOH. Controlled acidification of the corresponding sodium salt with acetic acid is very critical to obtain easy-filtering crystalline product in high yield and purity.

Procedure:

To the suspension of (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate VII (489.0 g, 91.9 Wt %, 633.3 mmol) in THF (1298 g) and MeOH (387 g) was added 50% NaOH (82.7 g, 949.9 mmol), followed by rinse with water (978 g). The mixture was stirred between 65-68° C. for about 1 h for complete hydrolysis. The resulting solution was cooled to 35° C., and filtered through an in-line filter (0.5 micron), and rinsed with a pre-mixed solution of water (978 g) and MeOH (387 g). The solution was heated to 60±4° C., and acetic acid (41.4 g, 689 mmol) was added over 1 h while the mixture was well agitated. The resulting suspension was stirred at 60±4° C. for 0.5 h. Another portion of acetic acid (41.4 g, 689 mmol) was charged in 0.5 h, and batch was stirred at 60±4° C. for additional 0.5 h. The batch was cooled to 26±4° C. over 1 h and held for 1 h. The batch was filtered, rinsed with a premixed solution of water (1956 g) and MeOH (773.6 g), dried at 50° C. under vacuum to give (E)-3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylic acid (1) (419.0 g, 95% yield) with ≧99.0 A % (240 nm) and 94.1 Wt % by HPLC.

### Step 11. Formation of Compound (1) Sodium Salt (Type A)

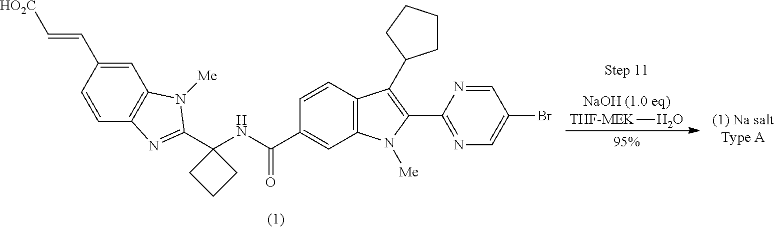

To a reactor were charged Compound (1) (150 g, mmol), THF (492 mL), H₂O (51 mL) and 45% aqueous NaOH solution (20.4 g, mmol). The mixture was stirred for >1 hr at ˜25° C. to form a clear solution (pH=9-11). To the solution was charged a suspension of Charcoal (1.5 g) and H₂O (27 mL). The mixture was stirred at ˜35° C. for >30 min and filtered. The filter was rinsed with THF (108 mL) and H₂O (21 mL). The filtrate was heated to 50° C. and charged with methyl ethylketone (MEK) (300 mL). The mixture was seeded with Compound (1) sodium salt MEK solvate (Type A) seeds (0.5 g) and stirred for another 1 hr at 50° C. To the mixture was charged additional MEK (600 mL). The resultant mixture was stirred for another 1 hr at 50° C. and then cooled to 25° C. The precipitate was filtered and rinsed with MEK twice (2×300 mL). The wet cake was dried in vacuum at 80° C. to give Compound (1) sodium salt (Type A) (145.6 g, 94%).

The Compound (1) sodium salt (Type A) MEK solvate seeds used in the above process step can be manufactured by the above process except without using seeds and without drying of the solvate.

Notes Regarding Crystallization Step 11

Process Optimization for Producing Higher Bulk Density Material

Observation of lab experiments showed that the seeding temperature should be reduced from 60° C. to 50° C. to prevent the dissolution of seed crystals. The crystallization kinetics in the THF/MEK/H₂O system was found to be slow, and oil/emulsion could be observed when anti-solvent MEK was added too fast after seeding. Thus experiments were performed to optimize the MEK addition time and aging time to minimize oiling. This improved process produced agglomerated granular crystals consistently that resulted in the desired high bulk density.

Optimization of Anti-Solvent Addition and Aging Time

An experiment was designed to optimize the aging time following the MEK anti-solvent addition at 50° C. The data indicated that all solids crystallized out of solution within 3 hours of aging. Following aging, the slurry was cooled linearly over 2 hours to 20° C. The extended aging time did not significantly improve yield losses in the mother liquor. The crystallization resulted in a 92.4% yield.

Immediately after the completion of the MEK addition, a milky oily solution was observed along with a large amount of crystals. The oily solution dissipated within one hour. A separate experiment determined that a slower addition rate of MEK can avoid the formation of oil.

The XRPD pattern on the wet cake confirmed the MEK solvated phase.

Another experiment was carried out to adapt the process for the slow crystallization kinetics observed in the current crystallization system. A ½ hour aging time was included after seeding and the MEK anti-solvent addition time was increased from 2 to 4 hours at 50° C.

All solids were found to have crystallized out of solution within 2 hours of aging. Following aging, the slurry was cooled linearly over 2 hours to 20° C. and held overnight. This did not improve on the mother liquor losses significantly.

In conclusion, the slurry at the end of the MEK addition was found to produce clear mother liquors without an oil phase; whereas previously in the 2-hour MEK addition, a milky oily mother liquor was observed. The recommendation is for a 4-hour anti-solvent addition to prevent the oiling.

Drying Time Study

A study was conducted to determine the required drying time at 80° C. to meet the ICH limits of residual solvents of MEK and THF. The results showed that drying for a minimum of 5 hours is required to meet the ICH limit on THF.

Effects of Water Content on Yield and Crystallization

The effect of water content on crystallization was evaluated. The water content was varied from the 5.6% (w/w) level specified in the existing procedure. The study was done using 50% more and 50% less water in the crystallization. The data indicated that 5.6% water content is near optimum for good yield and operability.

IV. Formulations of Compound (2)

Examples of pharmaceutical formulations containing Compound (2) include the tablet formulations described below.

### Example 5

### Solid Oral Formulation #1

The composition of the solid oral formulation:

| | Monograph | Functionality | % w/w |
|---|---|---|---|
| Compound (2) Na salt | | Active | 34.45 |
| Meglumine | USP/Ph. Eur. | Basifier | 7.00 |
| Sodium Lauryl Sulfate | NF/Ph. Eur. | Surfactant | 3.50 |
| Polyethylene Glycol 6000 | NF/Ph. Eur. | Solubilizer/Binder | 10.33 |
| Mannitol | USP/Ph. Eur. | Filler | 43.72 |
| Colloidal Silicon Dioxide | NF/Ph. Eur. | Glidant | 0.75 |
| Magnesium Stearate | NF/Ph. Eur. | Lubricant | 0.75 |

Two specific solid oral drug product formulations were prepared according to the above general Formulation #1, a 50 mg product and a 200 mg product.

| | | 200 mg | 50 mg |
|---|---|---|---|
| Ingredient | Function | mg/tablet | mg/tablet |
| Compound (2) Na salt¹ | Drug Substance | 206.7¹ | 51.7¹ |
| Meglumine | Basifier | 42.0 | 10.5 |
| Sodium Lauryl Sulfate | Surfactant | 21.0 | 5.3 |
| Polyethylene Glycol 6000 | Solubilizer Binder | 62.0 | 15.5 |
| Mannitol (powdered) | Filler | 262.3 | 65.6 |
| Purified Water² | Granulating agent | q.s. | q.s. |
| Colloidal Silicon Dioxide | Glidant | 3.0 | 0.8 |
| Magnesium Stearate³ | Lubricant | 3.0 | 0.8 |

| | | |
|---|---|---|
| Total | 600.0 | 150.0 |

¹206.7 mg and 51.7 mg Compound (2) Na salt (sodium salt) is equivalent to 200 mg and 50 mg of the active moiety, Compound (2) (free acid), respectively.
²Purified water is used as a granulating agent; it does not appear in the final product.
³Vegetable origin

### Example 6

### Solid Oral Formulation #2

The composition of the solid oral formulation:

| | Monograph | Functionality | % w/w |
|---|---|---|---|
| Compound (2) Na salt | | Active | 40.00 |
| Arginine | USP/Ph. Eur. | Basifier | 8.00 |
| Sodium Lauryl Sulfate | NF/Ph. Eur. | Surfactant | 4.00 |
| Polyethylene Glycol 8000 | NF/Ph. Eur. | Solubilizer/Binder | 12.00 |
| Mannitol | USP/Ph. Eur. | Filler | 35.00 |
| Colloidal Silicon Dioxide | NF/Ph. Eur. | Glidant | 0.50 |
| Magnesium Stearate | NF/Ph. Eur. | Lubricant | 0.50 |

Two specific solid oral drug product formulations were prepared according to the above general Formulation #1, a 200 mg product and a 400 mg product.

| | | 200 mg | 400 mg |
|---|---|---|---|
| Ingredient | Function | mg/tablet | mg/tablet |
| Compound (2) Na salt¹ | Drug Substance | 206.7¹ | 413.4¹ |
| Arginine | Basifier | 41.4 | 82.7 |
| Sodium Lauryl Sulfate | Surfactant | 20.7 | 41.3 |
| Polyethylene Glycol 8000 | Solubilizer/Binder | 62.0 | 124.0 |
| Mannitol (powdered) | Filler | 180.9 | 361.8 |
| Purified Water² | Granulating agent | q.s. | q.s. |
| Colloidal Silicon Dioxide | Glidant | 2.6 | 5.2 |
| Magnesium Stearate³ | Lubricant | 2.6 | 5.2 |

| | | |
|---|---|---|
| Total | 516.8 | 1033.6 |

¹206.7 mg and 413.4 mg Compound (2) Na salt (sodium salt) is equivalent to 200 mg and 400 mg of the active moiety, Compound (2) (free acid), respectively.
²Purified water is used as a granulating agent; it does not appear in the final product.
³Vegetable origin

Preparation of Formulations 1-2

The drug substance along with the intragranular excipients including the basifier, surfactant, solubilizer/binder, filler are mixed in a dry state in a high shear granulator prior to addition of water. The drug substance and the excipients may be screened prior to milling to remove large agglomerates if necessary. After mixing is complete, the mixture is granulated using purified water as a granulating agent in the high shear granulator till a suitable end point is achieved. The wet granules are removed and dried at appropriate drying temperatures either in a tray dryer or a fluid bed dryer. The dried granules are milled by passing through a high speed mill, such as a Comill. Milled granules are then blended with the extragranular excipients, glidant and lubricant and then tableted in a tablet press.

V. Clinical Results

For the clinical trials described below, the Compound (1) drug product was administered as a softgel capsule lipid-based formulation containing Compound (1) sodium salt. Compound (2) drug product was administered as a tablet formulation containing Compound (2) sodium salt.

### Example 7

### Clinical Study with Treatment-Naïve Patients

Safety and antiviral activity of interferon-sparing treatment with the protease inhibitor Compound (1) sodium salt, the HCV polymerase inhibitor Compound (2) sodium salt and ribavirin in treatment-naive patients with chronic hepatitis C genotype-1 infection.

Background: Compound (1) and Compound (2) are potent and specific inhibitors of the HCV NS3/4A protease and NS5B RNA-dependent RNA polymerase, respectively. An interferon-free combination of both antivirals with ribavirin (RBV) to eradicate HCV infection would create a major paradigm shift in HCV treatment.

Methods: In this randomized open-label trial, 32 treatment-naïve HCV genotype-1 patients were treated over 4 weeks with Compound (2) sodium salt given at 400 mg or 600 mg three times a day (TID), Compound (1) sodium salt given at 120 mg daily (QD) and RBV (weight based dosage at 1000 mg or 1200 mg daily in two doses). Plasma HCV RNA virus load (VL) was measured by Roche COBAS® TaqMan® assay with a lower limit of quantification of 25 IU/ml.

Results: At baseline, mean age was 51±11 years, mean BMI 23.8±3.5 kg/m², mean VL 6.48 LOG₁₀. All patients had a rapid and sharp VL decline during the first two days, followed by a slower second phase decline in all except 2 patients. One patient experienced VL breakthrough (increase by >1 LOG₁₀ from nadir during treatment) and one other experienced a 0.7 LOG₁₀ VL increase. Both were in the lower dose group (400 mg HD Compound (2)) and were genotype 1a patients (using the Trugen assay) with high baseline VL. On day 29, all patients were switched per protocol to treatment with Compound (1) sodium salt, and pegylated interferon(PegIFN)/RBV. Virological response rates (VL<25 IU/ml) after 1, 2, 3 and 4 weeks of oral treatment are shown in the table.

TABLE
Proportion of patients with viral load <25 IU/ml

| | Day 8 | Day 15 | Day 22 | Day 29 |
|---|---|---|---|---|
| 400 mg TID Cmpd (2) + | 4/15 | 6/15 | 10/15 | 11/15 |
| Cmpd (1) + RBV | | | | |
| 600 mg TID Cmpd (2) + | 3/17 | 14/17 | 17/17 | 17/17 |
| Cmpd (1) + RBV | | | | |

Below is a more detailed presentation of the same data showing virologic response by dose group and by subgenotype (where BLQ=VL<25 IU/ml):

| | Day 8 | Day 15 | Day 22 | Day 29 |
|---|---|---|---|---|
| | BLQ | BLQ | BLQ | BLQ |
| 400 mg TID | 4 | 6 | 10 | 11 |
| N = 15 | 27% | 40% | 67% | 73% |
| | 1a: 2/8 | 1a: 3/8 | 1a: 5/8 | 1a: 4/8 |
| | 1b: 2/6 | 1b: 3/6 | 1b: 5/6 | 1b: 6/6 |
| 600 mg TID | 3 | 14 | 17 | 17 |
| N = 17 | 18% | 82% | 100% | 100% |
| | 1a: 2/5 | 1a: 5/5 | 1a: 5/5 | 1a: 5/5 |
| | 1b: 1/9 | 1b: 6/9 | 1b: 9/9 | 1b: 9/9 |
| Total | 7 | 20 | 27 | 28 |
| N = 32 | 18% | 63% | 84% | 88% |
| | 1a: 4/13 | 1a: 8/13 | 1a: 10/13 | 1a: 9/13 |
| | 1b: 3/15 | 1b: 9/15 | 1b: 14/15 | 1b: 15/15 |

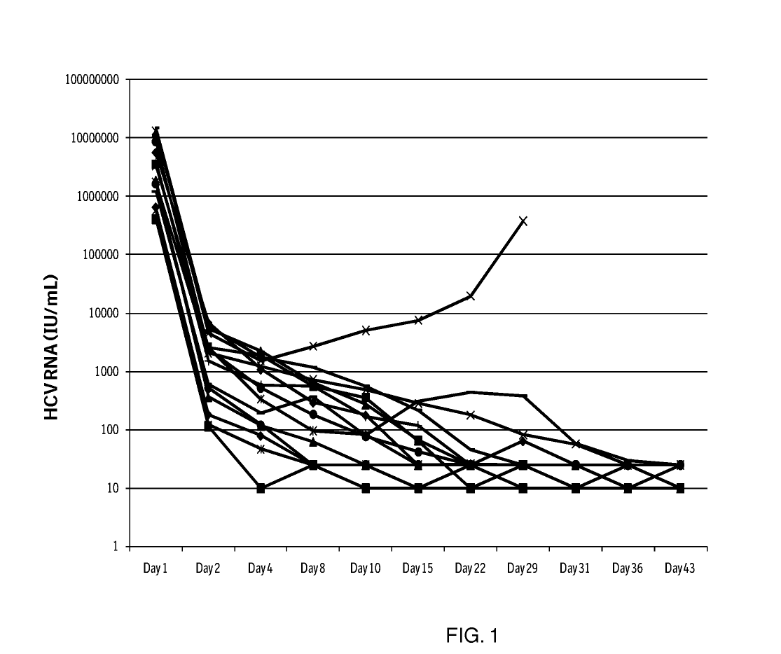

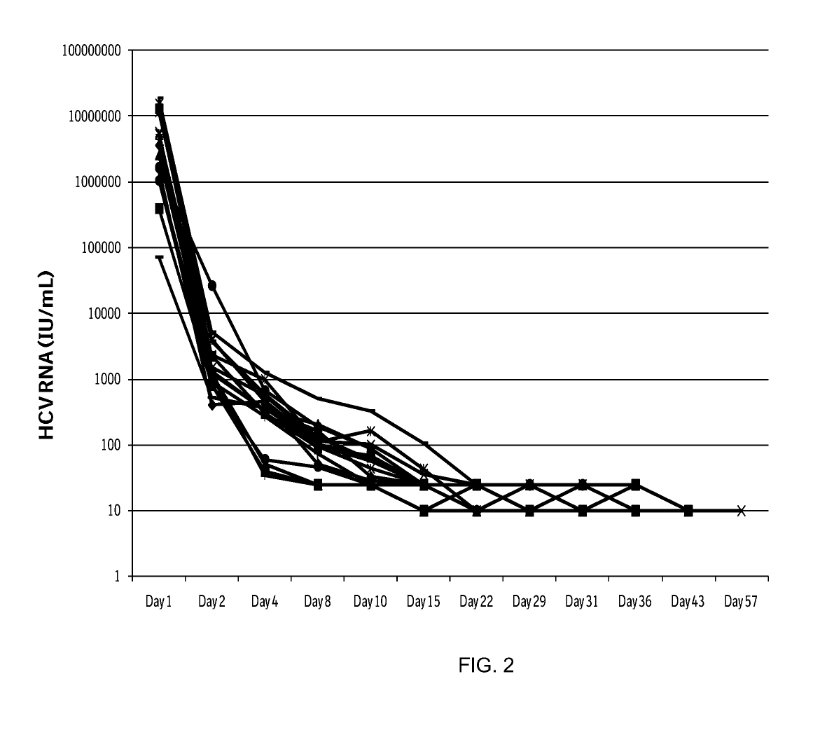

The results demonstrate robust antiviral activity at 400 mg TID (overall 73% RVR; 100% for GT1b and 50% for GT1a), and excellent antiviral activity at 600 mg TID (100% RVR for GT1a and 1b). The change in VL over time for the 400 mg TID dose group is graphically depicted in FIG. 1, and the change in VL over time for the 600 mg TID dose group is graphically depicted in FIG. 2.

At the higher dose level, there was no difference between genotype (GT) 1a and 1b, while GT1a patients at 400 mg TID had a lower response rate than those with GT1b, and one GT1a patient in the 400 mg TID dose group demonstrated a viral rebound (increase of ≧1−log₁₀ from nadir) during the 28-day treatment. The PegIFN sparing treatment was well tolerated. The most common adverse events (AEs) were mostly mild gastro-intestinal effects (diarrhea, nausea, vomiting), rashes or photosensitivity. There were no severe AEs, SAEs or treatment discontinuations within the 4-week study period. Laboratory parameters did not indicate any relevant changes from baseline, except for a continuous drop in ALT in all patients, a decrease of haemoglobin (median −2.5 and −3.6 g/dL) and increase of unconjugated bilirubin (median +9.8 and +11.5 umol/l).

In addition, based on feedback from investigators, the combination therapy was found as having improved tolerability as compared to other HCV treatments. A questionaire to compare tolerability of the triple combination therapy of the present invention vs. other HCV regimens was sent to all 14 investigators. Tolerability was rated on a scale from −5 to +5 (with “0” indicating comparable tolerability, “−5” much worse tolerability and “+5” much better tolerability).

The results are shown in the table below:

| | | | me- | | |
|---|---|---|---|---|---|
| Trial Description | vs. Other HCV Treatment | n | dian | min | max |
| Compound (1) + | vs. SOC (PegIFN/RBV) | 8 | +3.5 | +2 | +5 |
| Compound (2) + | vs. Compound (1) + SOC | 8 | +3.5 | 0 | +5 |
| RBV | vs. Compound (2) + SOC | 5 | +3 | 0 | +5 |
| | vs. Telaprevir + SOC | 7 | +4 | +3 | +5 |
| | vs. other PegIFN- | 2 | +2.5 | 0 | +5 |
| | sparing regimen | | | | |

### CONCLUSIONS

PegIFN sparing treatment with the NS3/4A inhibitor Compound (1), NS5B inhibitor Compound (2), and RBV demonstrated strong early antiviral activity against HCV genotype 1 with good safety and tolerability. A phase IIb trial testing different dose regimens of this combination, with longer durations, is planned to evaluate sustained virologic response rates.

Only one GT1a patient in the 400 mg TID dose group demonstrated a rebound in viral load during the 28-day treatment period. Sequencing of the viral nucleic acid from the rebound sample, showed that the virus contained sequence changes, relative to the baseline pre-treatment sample, in both the NS3 protease and NS5B polymerase regions. The nucleotide changes encoded for amino acid subsutitions R155K in NS3 and P495L in NS5B and represent virus that is dually resistant to compound (1) and compound (2). The low frequency of virologic rebound (only one out of 15 patients in the 400 mg TID dose group, and no patients out of 17 in the 600 mg TID dose group) during treatment is a surprising and unexpected result based on earlier assessment of compound (1) as a monotherapy in a PegIFN sparing regimen for 14-days where the vast majority of both 1a and 1b patients demonstrated virologic rebound during the treatment period (see the monotherapy data for Compound (1) presented in U.S. Application Publication 2010/0068182).

Definitely surprising results include the fact that the PegIFN sparing regimen comprised of compound (1), compound (2) and ribavirin effectively suppresses the emergence of drug resistant variants that have been commonly observed in clinical trials of compound 1 or other protease inhibitors in monotherapy. The low frequency of virologic failure ( 1/15) in the 400 mg TID dose group and the lack of any virology failures (0/17) in the 600 mg TID dose group suggest that the combination of compounds 1 and 2 have the potential to achieve significantly improved efficacies in a novel and more tolerable therapeutic regimen.

1. A method of treating hepatitis C viral (HCV) infection or alleviating one or more symptoms thereof in a patient comprising the step of administering to the patient a therapeutic combination comprising:
(a) a compound of the following formula (1) or a pharmaceutically acceptable salt thereof: 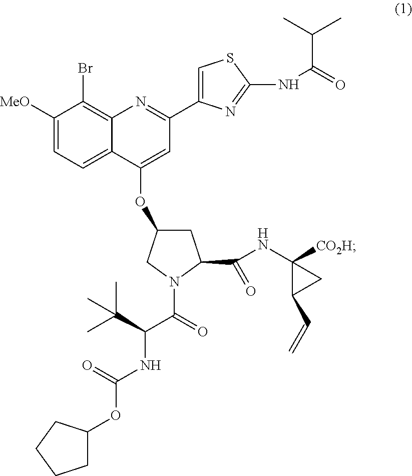
(b) a compound of the following formula (2) or a pharmaceutically acceptable salt thereof: 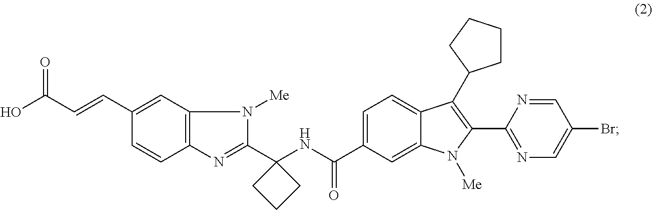
and optionally (c) ribavirin.

2. The method according to claim 1, wherein the HCV infection is genotype 1.

3. The method according to claim 1, wherein said patient is a treatment-naive patient.

4. The method according to claim 1, wherein said patient is non-responsive to a combination therapy using ribavirin and an interferon alpha.

5. The method according to claim 1, wherein the HCV-RNA levels of said patient are reduced to a level below 25 International Units (IU) per ml of serum or plasma as a result of the treatment.

6. The method according to claim 1, wherein said therapeutic combination is administered for at least 4 weeks.

7. The method according to claim 1, wherein compound (1) or a pharmaceutically acceptable salt thereof is administered at a dosage between about 40 mg per day and about 480 mg per day.

8. The method according to claim 1, wherein compound (1) or a pharmaceutically acceptable salt thereof is administered at a dosage between about 120 mg per day and about 240 mg per day.

9. The method according to claim 1, wherein compound (1) is administered in the form of its sodium salt.

10. The method according to claim 1, wherein compound (2) or a pharmaceutically acceptable salt thereof is administered at a dosage between about 800 mg per day and about 2400 mg per day.

11. The method according to claim 1, wherein compound (2) or a pharmaceutically acceptable salt thereof is administered at a dosage between about 1200 mg per day and about 1800 mg per day.

12. The method according to claim 1, wherein compound (2) is administered in the form of its sodium salt.

13. The method according to claim 1, wherein said ribavirin is administered at a dosage between about 400 mg/day and about 1200 mg/day.

14. The method according to claim 1, wherein said ribavirin is administered at a dosage between about 1000 mg/day and about 1200 mg/day.

15. The method according to claim 1, wherein the therapeutic combination administered is a triple combination therapy including administration of Compound (1) or a pharmaceutically acceptable salt thereof, Compound (2) or a pharmaceutically acceptable salt thereof and ribavirin.

16. The method according to claim 1, wherein the therapeutic combination administered is a double combination therapy including administration of Compound (1) or a pharmaceutically acceptable salt thereof and Compound (2) or a pharmaceutically acceptable salt thereof without the administration of ribavirin.

17. The method according to claim 1, wherein the therapeutic combination administered comprises:
(a) Compound (1) or a pharmaceutically acceptable salt thereof at a dosage between about 120 mg/day to about 240 mg/day;
(b) Compound (2) or a pharmaceutically acceptable salt thereof at a dosage between about 1200 mg/day to about 1800 mg/day; and
(c) optionally ribavirin at a dosage of between about 1000 mg/day to about 1200 mg/day.

18. A packaged pharmaceutical composition comprising a packaging containing:
(a) one or more doses of the following Compound (1) or a pharmaceutically acceptable salt thereof: 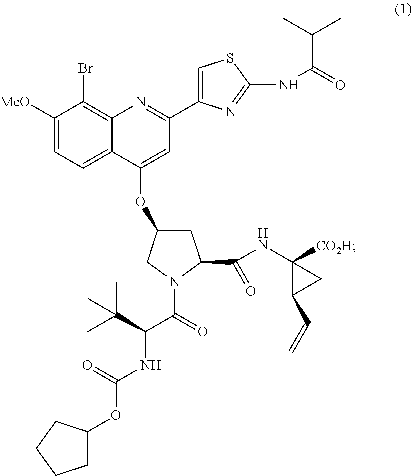
or (b) one or more doses of the following Compound (2) or a pharmaceutically acceptable salt thereof: 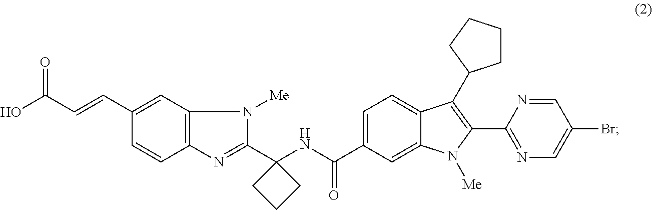
and written instructions directing the co-administration of Compound (1), or a pharmaceutically acceptable salt thereof, and Compound (2), or a pharmaceutically acceptable salt thereof, and optionally ribavirin for the treatment of HCV infection.

19. A kit for the treatment of HCV infection comprising:
(a) one or more doses of the following Compound (1) or a pharmaceutically acceptable salt thereof: 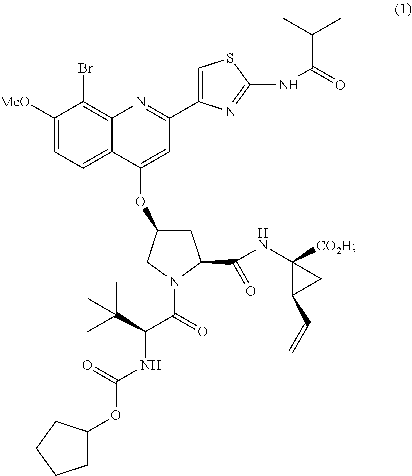
and (b) one or more doses of the following Compound (2) or a pharmaceutically acceptable salt thereof: 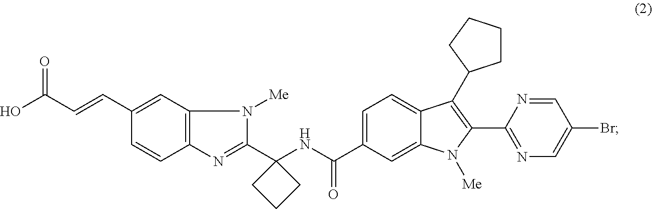
and written instructions directing the co-administration of Compound (1), or a pharmaceutically acceptable salt thereof, and Compound (2), or a pharmaceutically acceptable salt thereof, and optionally ribavirin for the treatment of HCV infection.
